(19) United States
(12) Patent Application Publication
(10) Pub. No.: US 2024/0294967 A1
(43) Pub. Date: Sep. 5, 2024

(54) METHODS OF DETECTING METHYLCYTOSINE AND HYDROXYMETHYLCYTOSINE BY SEQUENCING

(71) Applicant: Illumina, Inc., San Diego, CA (US)

(72) Inventors: Xiaolin WU, Cambridge (GB); Antoine FRANCAIS, Cambridge (GB); Xiaohai LIU, Cambridge (GB)

(21) Appl. No.: 18/569,532

(22) PCT Filed: Jan. 18, 2023
(86) PCT No.: PCT/US2023/011047

§ 371 Date: Dec. 12, 2023

Related U.S. Application Data
(60) Provisional application No. 63/301,370, filed on Jan. 20, 2022.

(51) Int. Cl. C12Q 1/6806; C12Q 1/26; C12Q 1/48; C12Q 1/6869

(52) U.S. Cl. CPC C12Q 1/6806; C12Q 1/26; C12Q 1/48; C12Q 1/6869

(57) ABSTRACT

Embodiments of the present disclosure relates to various bisulfite-free chemical methods for detecting methylation of cytosine in the DNA sample. These methods convert methylated and hydroxymethylated cytosine in the nucleic acid sequence to a modified or pseudo thymine or a uracil moiety which then can be detected in sequencing.

### BACKGROUND

### Field

The present disclosure relates to compositions and methods for detecting methylation of cytosine in the DNA sample by sequencing.

### Description of the Related Art

In the human genome, the most prevalent modified base is mC, which accounts for about 1-5% of all nucleobases in the genome. Cytosine methylation occurs throughout the whole genome and is generally associated with transcriptional repression, although in some cases it can have the opposite effect. In somatic cells, mC is found primarily at CpG sites—of which 60-80% are symmetrically methylated. Additionally, in embryonic stem cells, where mC level are generally more elevated, significant non-CpG methylations have been observed. These epigenetic modifications are of a clinical significance.

Bisulfite sequencing has been the gold standard for mapping DNA modifications including 5-methylcytosine (5mC) and 5-hydroxymethylcytosine (5hmC). Bisulfite sequencing relies on the complete conversion of unmodified cytosine to thymine leaving 5mC and 5hmC untouched. However, the harsh bisulfite treatment causes severe degradations of DNA due to the acidic conditions. Converting all these positions to thymine severely reduces sequence complexity (3 base A/G/T sequencing), leading to poor sequencing quality, low mapping rates, uneven genome coverage. Alternative bisulfite-free chemistries involving the use of TET-assisted pyridine borane for detecting 5mC and 5hmC in DNA sample and the use of peroxogungstate for detecting 5mC and 5hmC in RNA samples have recently been reported by Liu et al., Nature Biotechnology 2019, 37, 424-429 and Yuan et al., Chem Commun 2019, 55, 2328-2331 respectively. However, these methods usually require larger sample input and have not proved to be successful for sensitive low-input samples, such as circulating cell-free DNA and single-cell analysis.

Therefore, there remains a challenge and a need for developing a sample preparative method that are compatible with sequencing, in particular sequencing by synthesis (SBS). Described herein are several bisulfite-free methods for selectively converting mC and hmC into a T equivalent or an alternative base. The methods described herein may prevent severe DNA damage and retain the similar genome coverage of A/C/G/T.

### SUMMARY

One aspect of the present disclosure relates to a method of identifying one or more hydroxymethylated cytosines of a nucleic acid sequence in a nucleic acid sample, comprising:
contacting the nucleic acid sample with a composition comprising an oxidative reagent;
converting the hydroxymethylated cytosines to modified thymine moieties each having the structure of Formula (I) or (II):

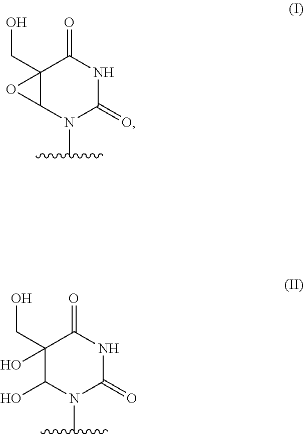 [SMILES: *N1C(=O)NC(=O)C2(CO)OC12 ; *N1C(=O)NC(=O)C(O)(CO)C1O]

to form a modified nucleic acid sequence; and
amplifying the modified nucleic acid sequence. In some embodiments, the method further comprises: sequencing the amplified modified nucleic acid sequence; and determining the sites of the modified thymine moiety by comparing the modified nucleic acid sequence to a reference nucleic acid sequence.

Another aspect of the present disclosure relates to a method of identifying cytosine methylation of a nucleic acid sequence in a nucleic acid sample, comprising:
contacting the nucleic acid sample with a TET enzyme to convert methylated cytosines to hydroxymethylated cytosines in the nucleic acid sequence;
reacting the TET treated nucleic acid sample with a composition comprising an oxidative reagent to convert the hydroxymethylated cytosines to modified thymine moieties each having the structure of Formula (I) or (II):

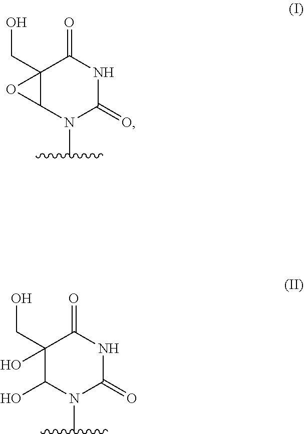 [SMILES: *N1C(=O)NC(=O)C2(CO)OC12 ; *N1C(=O)NC(=O)C(O)(CO)C1O]

to form a modified nucleic acid sequence; and
amplifying the modified nucleic acid sequence. In some embodiments, the method further comprises: sequencing the amplified modified nucleic acid sequence; and determining the sites of the modified thymine moieties by comparing the modified nucleic acid sequence to a reference nucleic acid sequence.

Some aspect of the present disclosure relates to a method of identifying one or more hydroxymethylated cytosines of a nucleic acid sequence in a nucleic acid sample, comprising:
contacting the nucleic acid sample with

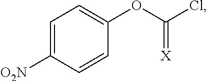

wherein X is O or S;
converting the hydroxymethylated cytosines to pseudo thymine moieties each having the structure of Formula (IIIa) or (IIIb):

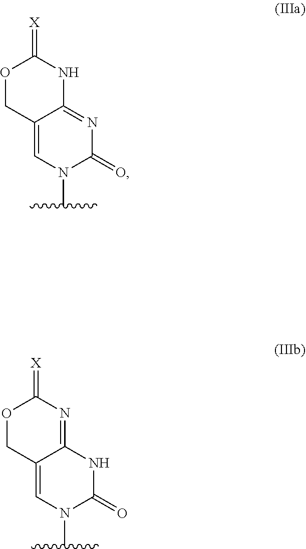

to form a modified nucleic acid sequence; and
amplifying the modified nucleic acid sequence. In some embodiments, the method further comprises: sequencing the amplified modified nucleic acid sequence; and determining the sites of the pseudo thymine moiety by comparing the modified nucleic acid sequence to a reference nucleic acid sequence.

Another aspect of the present disclosure relates to a method of identifying cytosine methylation of a nucleic acid sequence in a nucleic acid sample, comprising:
contacting the nucleic acid sample with a TET enzyme to convert methylated cytosines to hydroxymethylated cytosines in the nucleic acid sequence;
reacting the TET treated nucleic acid sample with

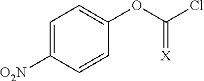

to convert the hydroxymethylated cytosines to pseudo thymine moieties each having the structure of Formula (IIIa) or (IIIb):

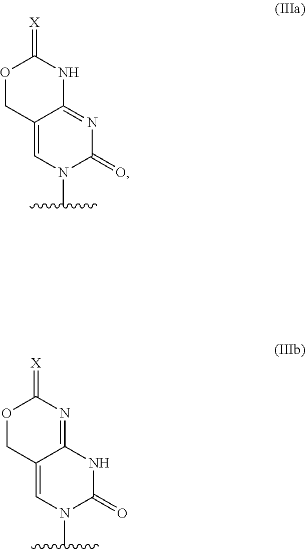

to form a modified nucleic acid sequence, wherein X is O or S; and
amplifying the modified nucleic acid sequence. In some embodiments, the method further comprises: sequencing the amplified modified nucleic acid sequence; and determining the sites of the pseudo thymine moieties by comparing the modified nucleic acid sequence to a reference nucleic acid sequence.

A further aspect of the present disclosure relates to a method of identifying cytosine methylation of a nucleic acid sequence in a nucleic acid sample, comprising:
contacting the nucleic acid sample with a TET enzyme to convert methylated and hydroxymethylated cytosines in the nucleic acid sequence to carboxylated cytosines;
reacting the TET treated nucleic acid sample with a cyanate or thiocyanate to convert the carboxylated cytosines to pseudo thymine moieties each having the structure of Formula (IIId):

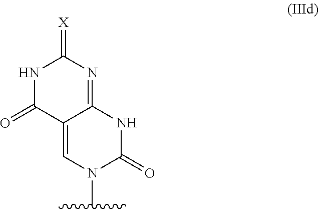

to form a modified nucleic acid sequence, wherein X is O or S; and
amplifying the modified nucleic acid sequence. In some embodiments, the method further comprises: sequencing the amplified modified nucleic acid sequence; and determining the sites of pseudo thymine moieties by comparing the modified nucleic acid sequence to a reference nucleic acid sequence.

Some aspect of the present disclosure relates to a method of identifying one or more hydroxymethylated cytosine of a nucleic acid sequence in a nucleic acid sample, comprising:
contacting the nucleic acid sample with

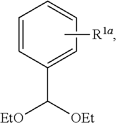

wherein R^{1a} is an optionally present hydrophilic electron withdrawing group;
converting the hydroxymethylated cytosines to pseudo thymine moieties having the structure of Formula (IVb):

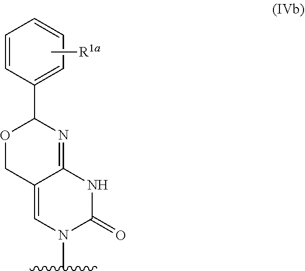

to form a modified nucleic acid sequence; and
amplifying the modified nucleic acid sequence. In some embodiments, the method further comprises: sequencing the amplified modified nucleic acid sequence; and determining the sites of the pseudo thymine moiety by comparing the modified nucleic acid sequence to a reference nucleic acid sequence.

Another aspect of the present disclosure relates to a method of identifying cytosine methylation of a nucleic acid sequence in a nucleic acid sample, comprising:
contacting the nucleic acid sample with a TET enzyme to convert methylated cytosines to hydroxymethylated cytosines in the nucleic acid sequence;
reacting the TET treated nucleic acid sample with

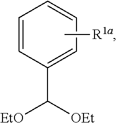

to convert the hydroxymethylated cytosines to pseudo thymine moieties having the structure of Formula (IVb):

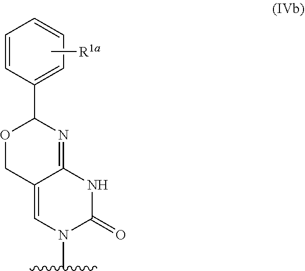

to form a modified nucleic acid sequence, wherein R^{1a} is a an optionally present hydrophilic electron withdrawing group; and
amplifying the modified nucleic acid sequence. In some embodiments, the method further comprises: sequencing the amplified modified nucleic acid sequence; and determining the sites of the pseudo thymine moieties by comparing the modified nucleic acid sequence to a reference nucleic acid sequence.

A further aspect of the present disclosure relates to a method of identifying cytosine methylation of a nucleic acid sequence in a nucleic acid sample, comprising:
contacting the nucleic acid sample with a TET enzyme to convert methylated and hydroxymethylated cytosines in the nucleic acid sequence to carboxylated cytosines;
reacting the TET treated nucleic acid sample first with ammonia in the presence of a carboxyl activating agent (e.g., DCC or EDC), then reacting with

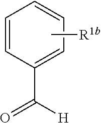

to convert carboxylated cytosines to pseudo thymine moieties each having the structure of Formula (IVd):

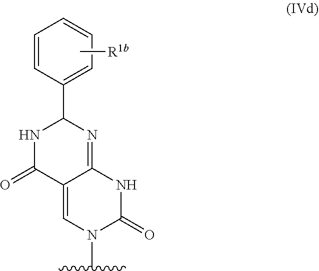

to form a modified nucleic acid sequence, wherein R^{1b} is an optionally present hydrophilic group; and
amplifying the modified nucleic acid sequence. In some embodiments, the method further comprises: sequencing the amplified modified nucleic acid sequence; and determining the sites of the pseudo thymine moieties by comparing the modified nucleic acid sequence to a reference nucleic acid sequence.

Some aspect of the present disclosure relates to a method of identifying cytosine methylation of a nucleic acid sequence in a nucleic acid sample, comprising:
contacting the nucleic acid sample with a TET enzyme to convert methylated and hydroxymethylated cytosines in the nucleic acid sequence to carboxylated cytosines;
reacting the TET treated nucleic acid sample with

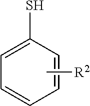

in a Michael Addition reaction to convert the carboxylated cytosines to first intermediates each having the structure of Formula (Va):

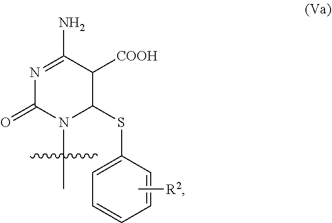

wherein R² is 4-OCH₃, 4-CH₃, 2-OCH₃, 4-Cl, 4-NO₂, or 4-CF₃;
treating the first intermediates with hydrogen peroxide to form second intermediates each having the structure of Formula (Vb):

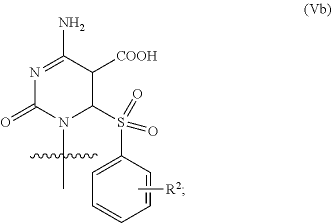

reacting the second intermediates with 1,8-diazabicyclo[5.4.0]undec-7-ene (DBU) to convert the second intermediates to uracil moieties to form a modified nucleic acid sequence; and
amplifying the modified nucleic acid sequence. In some embodiments, the method further comprises: sequencing the amplified modified nucleic acid sequence; and determining the sites of the converted uracil moieties by comparing the modified nucleic acid sequence to a reference nucleic acid sequence.

Another aspect of the present disclosure relates to a method of identifying methylated cytosines of a nucleic acid sequence in a nucleic acid sample, comprising:
contacting the nucleic acid sample with β-glucosyltransferase (β-GT) to selectively glucosylating hydroxymethyl cytosines of the nucleic acid sequence;
contacting the β-GT treated nucleic acid sample with a TET enzyme to convert methylated cytosines in the nucleic acid sequence to carboxylated cytosines;
reacting the TET treated nucleic acid sample with

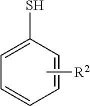

in a Michael Addition reaction to convert carboxylated cytosines to first intermediates each having the structure of Formula (Va):

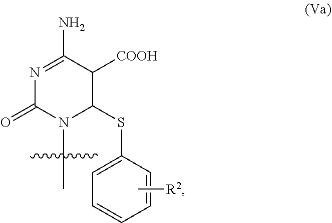

wherein R² is 4-OCH₃, 4-CH₃, 2-OCH₃, 4-Cl, 4-NO₂, or 4-CF₃;
treating the first intermediates with hydrogen peroxide to form second intermediates each having the structure of Formula (Vb):

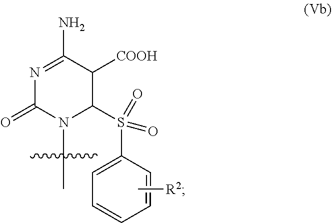

reacting the second intermediates with 1,8-diazabicyclo[5.4.0]undec-7-ene (DBU) to convert the second intermediates to uracil moieties to form a modified nucleic acid sequence; and
amplifying the modified nucleic acid sequence. In some embodiments, the method further comprises: sequencing the amplified modified nucleic acid sequence; and determining the sites of the converted uracil moieties by comparing the modified nucleic acid sequence to a reference nucleic acid sequence.

A further aspect of the present application relates to a method of identifying cytosine methylation of a nucleic acid sequence in a nucleic acid sample, comprising:
contacting the nucleic acid sample with a TET enzyme to convert methylated and hydroxymethylated cytosines in the nucleic acid sequence to carboxylated cytosines;
reacting the TET treated nucleic acid sample with an unsaturated reagent in a cycloaddition reaction to convert the carboxylated cytosines to first intermediates each having the structure of Formula (VI):

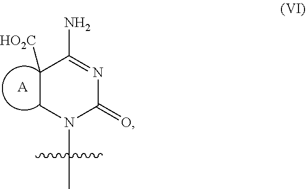

wherein ring A is an optionally substituted 4, 5 or 6 membered carbocyclyl or heterocyclyl ring;
converting the first intermediates to bicyclic thymine moieties each having a structure of Formula (VII):

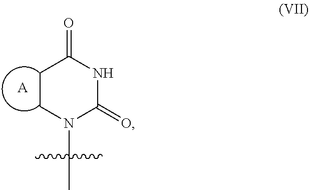

to form a modified nucleic acid sequence; and
amplifying the modified nucleic acid sequence. In some embodiments, the method further comprises: sequencing the amplified modified nucleic acid sequence; and determining the sites of the bicyclic thymine moieties by comparing the modified nucleic acid sequence to a reference nucleic acid sequence.

A further aspect of the present application relates to a method of identifying methylated cytosines of a nucleic acid sequence in a nucleic acid sample, comprising:
contacting the nucleic acid sample with β-glucosyltransferase (β-GT) to selectively glucosylating hydroxymethyl cytosines of the nucleic acid sequence;
contacting the β-GT treated nucleic acid sample with a TET enzyme to convert methylated cytosines in the nucleic acid sequence to carboxylated cytosines;
reacting the TET treated nucleic acid sample with an unsaturated reagent in a cycloaddition reaction to convert carboxylated cytosines to first intermediates each having the structure of Formula (VI):

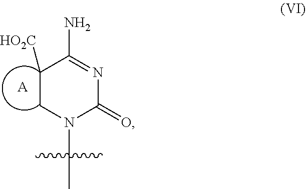

wherein ring A is an optionally substituted 4, 5 or 6 membered carbocyclyl or heterocyclyl ring;
converting the first intermediates to bicyclic thymine moieties each having a structure of Formula (VII):

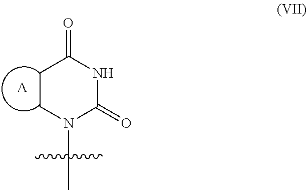

to form a modified nucleic acid sequence; and
amplifying the modified nucleic acid sequence. In some embodiments, the method further comprises: sequencing the amplified modified nucleic acid sequence; and determining the sites of the bicyclic thymine moieties by comparing the modified nucleic acid sequence to a reference nucleic acid sequence.

In any embodiments of the methods described herein, the nucleic acid sample may comprise or is a genomic DNA sample.

### BRIEF DESCRIPTION OF THE DRAWINGS

FIG. 1 illustrates the identification hydroxymethyl cytosine and cytosine methylation by using various chemistry conversion methods in conjunction with TET to convert hydroxymethyl cytosine and methyl cytosines to modified or pseudo thymine moieties according to several embodiments of the present application.

FIG. 2 illustrates the identification hydroxymethyl cytosine and cytosine methylation by using various chemistry conversion methods in conjunction with TET and β-glucosyltransferase to convert hydroxymethyl cytosine and methyl cytosines to uracil or bicyclic thymine moieties according to several embodiments of the present application.

### DETAILED DESCRIPTION

Embodiments of the present application relates to several bisulfite-free methods for mapping nucleic acid modifications (e.g., DNA methylations) without harsh chemical treatment to the nucleic acid sample. In particular, the methods described herein may selectively converting a hydroxymethyl cytosine (5bmC) and/or methyl cytosine (5mC) to a modified or pseudo thymine moiety or a uracil moiety, without affecting unmodified cytosines. The chemical modified nucleic acid sample may be directly used in sequencing (e.g., SBS) with high sensitivity and specificity. 5 mC and 5hmC are the two most common epigenetic marks found in the mammalian genome. Aberrant DNA methylation and hydroxymethylation have been associated with various diseases and are well accepted hallmarks of cancer. Therefore, effective methods described herein for determination of genomic distribution of 5mC and 5hmC are not only important for understanding of development of homeostatic, but also invaluable for clinical applications.

### Definitions

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

Where a range of values is provided, it is understood that the upper and lower limit, and each intervening value between the upper and lower limit of the range is encompassed within the embodiments.

As used herein, common organic abbreviations are defined as follows:
° C. Temperature in degrees Centigrade
mC or 5mC 5-methyl cytosine
hmc or 5hmc 5-hydroxymethyl cytosine
caC or 5caC 5-carboxycytosine
fC pr 5fC 5-formylcytosine
DCC N,N′-dicyclohexylcarbodiimide
EDC l-ethyl-3-(3-dimethylaminopropyl)carbodiimide
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
SBS Sequencing by Synthesis
TET enzyme Ten-eleven translocation methylcytosine dioxygenase
β-GT beta glycosyltransferase

As used herein, the term “methylated cytosine”, “mC” or “5mC” refers to 5-methyl cytosine having the structure:

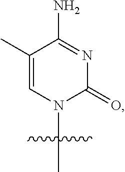 [SMILES: *n1cc(C)c(N)nc1=O]

which is attached to the ribose or 2-deoxyribose ring of a nucleoside or nucleotide.

As used herein, the term “hydroxymethylated cytosine”, “hmC” or “5hmC” refers to 5-hydroxymethyl cytosine having the structure:

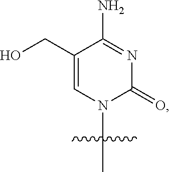 [SMILES: *n1cc(CO)c(N)nc1=O]

which is attached to the ribose or 2-deoxyribose ring of a nucleoside or nucleotide.

As used herein, the term “caC” or “5caC” refers to 5-carboxy cytosine having the structure:

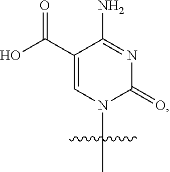 [SMILES: *n1cc(C(=O)O)c(N)nc1=O]

which is attached to the ribose or 2-deoxyribose ring of a nucleoside or nucleotide.

As used herein, the term “fC” or “5fC” refers to 5-formyl cytosine having the structure:

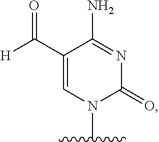 [SMILES: *n1cc(C=O)c(N)nc1=O]

which is attached to the ribose or 2-deoxyribose ring of a nucleoside or nucleotide.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Cₐ to C_{b}” in which “a” and “b” are integers refer to the number of carbon atoms in an alkyl, alkenyl or alkynyl group, or the number of ring atoms of a cycloalkyl or aryl group That is, the alkyl, the alkenyl, the alkynyl, the ring of the cycloalkyl, and ring of the aryl can contain from “a” to “b”, inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C₃, C₄, C₅ and C₆, and a range defined by any of the two numbers. For example, C₁-C₆ alkyl includes C₁, C₂, C₃, C₄, C₅ and C₆ alkyl, C₂-C₆ alkyl, C₁-C₃ alkyl, etc. Similarly, C₂-C₆ alkenyl includes C₂, C₃, C₄, C₅ and C₆ alkenyl, C₂-C₅ alkenyl, C₃-C₄ alkenyl, etc.; and C₂-C₆ alkynyl includes C₂, C₃, C₄, C₅ and C₆ alkynyl, C₂-C₅ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁-C₄alkyl” or similar designations. By way of example only, “C₁-C₆ alkyl” indicates that there are one to six carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula-OR wherein R is an alkyl as is defined above, such as “C₁-C₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂-C₆ alkenyl” or similar designations. By way of example only, “C₂-C₆ alkenyl” indicates that there are two to six carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2, -dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 6 carbon atoms. The alkynyl group may be designated as “C₂-C₆ alkynyl” or similar designations. By way of example only, “C₂-C₆ alkynyl” indicates that there are two to six carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyL, pentynyl, and hexynyl, and the like.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphtbyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁-C₆ alkylene group).

As used herein, “aryloxy” refers to RO— in which R is an aryl, as defined above, such as but not limited to phenyl.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinolinyl, benzoimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁-C₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, “—O-alkoxyalkyl” or “—O(alkoxy)alkyl” refers to an alkoxy group connected via an —O-(alkylene) group, such as —O—(C₁-C₆ alkoxy)C₁-C₆ alkyl, for example, —O(CH₂)₁₋₃—OCH₃.

As used herein, “haloalkyl” refers to an alkyl group in which one or more of the hydrogen atoms are replaced by a halogen (e.g., mono-haloalkyl, di-haloalkyl, and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy and 1-chloro-2-fluoromethoxy, 2-fluoroisobutoxy. A haloalkoxy may be substituted or unsubstituted.

An “amino” group refers to a —NH₂ group. The term “mono-substituted amino group” as used herein refers to an amino (—NH₂) group where one of the hydrogen atom is replaced by a substituent. The term “di-substituted amino group” as used herein refers to an amino (—NH₂) group where each of the two hydrogen atoms is replaced by a substituent. The term “optionally substituted amino,” as used herein refer to a —NR_{A}R_{B} group where R_{A} and R_{B} are independently hydrogen, alkyl, cycloalkyl, aryl, heteroaryl, heterocyclyl, aralkyl, or heterocyclyl(alkyl), as defined herein.

An “O-carboxy” group refers to a “—OC(═O)R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-carboxy” group refers to a “—C(═O)OR” group in which R is selected from the group consisting of hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes carboxyl (i.e., —C(═O)OH).

A “sulfonyl” group refers to an “—SO₂R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “S-sulfonamido” group refers to a “—SO₂NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-sulfonamido” group refers to a “—N(R_{A})SO₂R_{B}” group in which R_{A} and R_{b} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “O-carbamyl” group refers to a “—OC(═O)N(R_{A}R_{B})” group in which R_{A} and R_{B} can be the same as defined with respect to S-sulfonamido. An O-carbamyl may be substituted or unsubstituted.

An “N-carbamyl” group refers to an “ROC(═O)N(R_{A})-” group in which R and R_{A} can be the same as defined with respect to N-sulfonamido. An N-carbamyl may be substituted or unsubstituted.

An “O-thiocarbamyl” group refers to a “—OC(═S)—N(R_{A}R_{B})” group in which R_{A} and R_{B} can be the same as defined with respect to S-sulfonamido. An O-thiocarbamyl may be substituted or unsubstituted.

An “N-thiocarbamyl” group refers to an “ROC(═S)N(R_{A})-” group in which R and R_{A} can be the same as defined with respect to N-sulfonamido. An N-thiocarbamyl may be substituted or unsubstituted.

The term “hydroxy” as used herein refers to a —OH group.

The term “cyano” group as used herein refers to a “—CN” group.

The term “azido” as used herein refers to a —N₃ group.

When a group is described as “optionally substituted” it may be either unsubstituted or substituted. Likewise, when a group is described as being “substituted”, the substituent may be selected from one or more of the indicated substituents. As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇ carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₈ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), (aryl)C₁-C₆ alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), (5-10 membered heteroaryl)C₁-C₆ alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, (C₁-C₆ alkoxy)C₁-C₆ alkyl, —O(C₁-C₆ alkoxy)C₁-C₆ alkyl; (C₁-C₆ haloalkoxy)C₁-C₆ alkyl; —((C₁-C₆ haloalkoxy)C₁-C₆ alkyl; aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfonate (—SO₃), sulfate, sulfino, —OSO₂C₁-alkyl, monophosphate, diphosphate, triphosphate, and oxo (═O). Wherever a group is described as “optionally substituted” that group can be substituted with the above substituents.

When a compound is shown as charged (i.e., bearing one or more positive or negative charges), it is understood that the compound may also contain one or more anions or cations such that the compound is in neutral form.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e. a sugar lacking a hydroxy group that is present in ribose. The nitrogen containing heterocyclic base can be purine or pyrimidine base. Purine bases include adenine (A) and guanine (G), and modified derivatives or analogs thereof, such as 7-deaza adenine or 7-deaza guanine. Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleoside” is structurally similar to a nucleotide, but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art, and includes its tautomers. Similarly, the term “pyrimidine base” is used herein in its ordinary sense as understood by those skilled in the art, and includes its tautomers. A non-limiting list of optionally substituted purine-bases includes purine, adenine, guanine, deazapurine, 7-deaza adenine, 7-deaza guanine, hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” or “incorporating” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, “derivative” or “analog” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, phosphoranilidate and phosphoramidate linkages. “Derivative”, “analog” and “modified” as used herein, may be used interchangeably, and are encompassed by the terms “nucleotide” and “nucleoside” defined herein.

As used herein, the term “phosphate” is used in its ordinary sense as understood by those skilled in the art, and includes its protonated forms (for example,

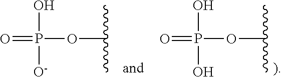 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The terms “protecting group” and “protecting groups” as used herein refer to any atom or group of atoms that is added to a molecule in order to prevent existing groups in the molecule from undergoing unwanted chemical reactions. Sometimes, “protecting group” and “blocking group” can be used interchangeably.

### Method of Methylation Detection by Oxidation of 5-Hydroxymethyl Cytosine

One aspect of the present disclosure relates to a method of identifying one or more hydroxymethylated cytosines (hmC) of a nucleic acid sequence in a nucleic acid sample, comprising:
contacting the nucleic acid sample with a composition comprising an oxidative reagent;
converting the hydroxymethylated cytosines to modified thymine moieties each having the structure of Formula (I) or (II):

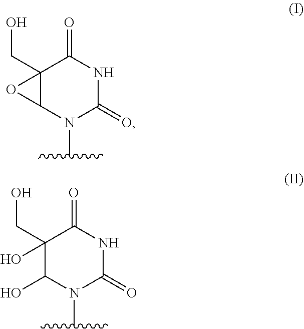

to form a modified nucleic acid sequence; and
amplifying the modified nucleic acid sequence.

In some embodiments, the oxidative reagent reacts with hydroxymethylated cytosine to form an epoxidation or a dihydroxylation intermediate, and the method further comprises hydrolyzing the epoxidation or dihydroxylation intermediate to form the modified thymine moiety. In this method, the methylation chemistries leverage the hydroxymethyl moiety of hmC. In particular, hydroxymethyl moiety will be used as a handle to direct oxidation specifically on the 5, 6 double bond of the cytosine. Different metal may be used to coordinate to the hydroxy group and perform dihydroxylation or epoxidation. Resulted intermediate may undergo hydrolysis resulting at the conversion to a modified thymine moiety (T*). The reaction scheme is illustrated in Scheme 1 below. The hmC is attached to a 2-deoxyribose ring of the nucleoside or nucleotide, which may be part of an oligonucleotide, a polynucleotide, or a nucleic acid sequence.

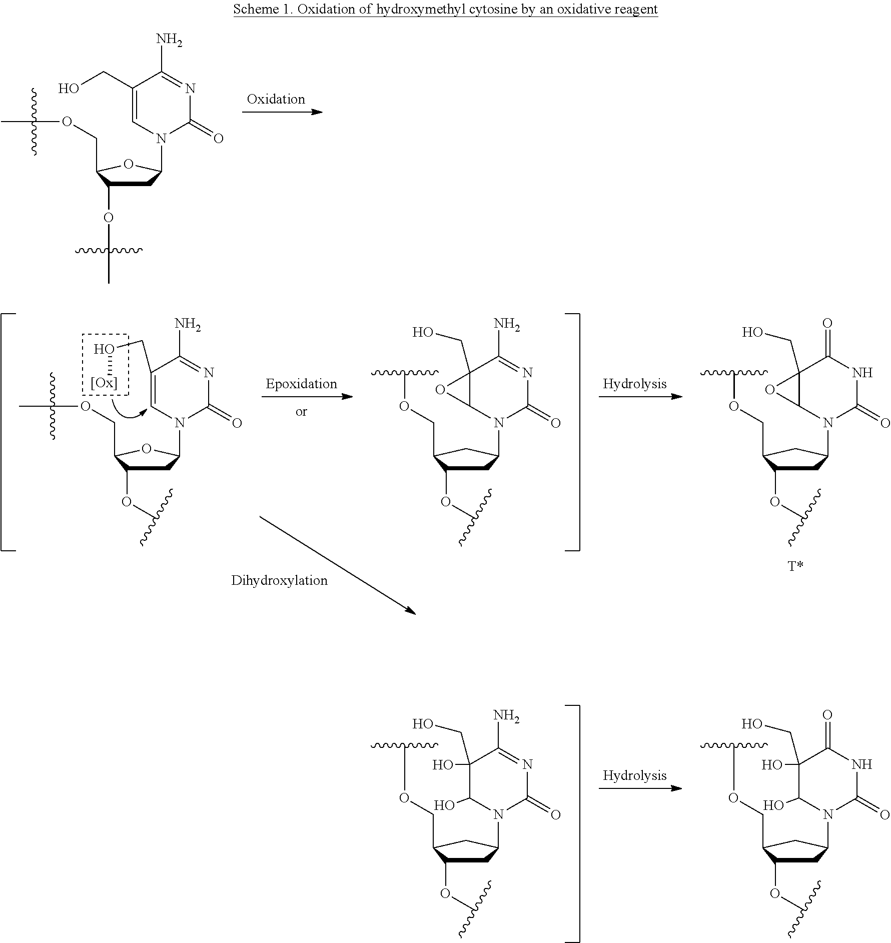

A variety of non-metallic or metallic oxidative agents may be used to perform this transformation. In some embodiments, the oxidative reagent comprises or is a peracid, for example, MPPA, or m-CPBA or a combination thereof. As a non-limiting example, the use of MPPA or m-CPBA is depicted in Scheme 2. hmC will be converted to the dehydroxylated C*, in which the aromatic system of nucleobase is broken. Subsequent hydrolysis will give epoxy T*, which will be converted to T by subsequent PCR during the library amplification. Oxidation with MPPA may be performed at room temperature in the presence of 0.5 M NaHCO₃ solution, while oxidation with m-CPBA may be performed at a mild basic environment of pH about 9.

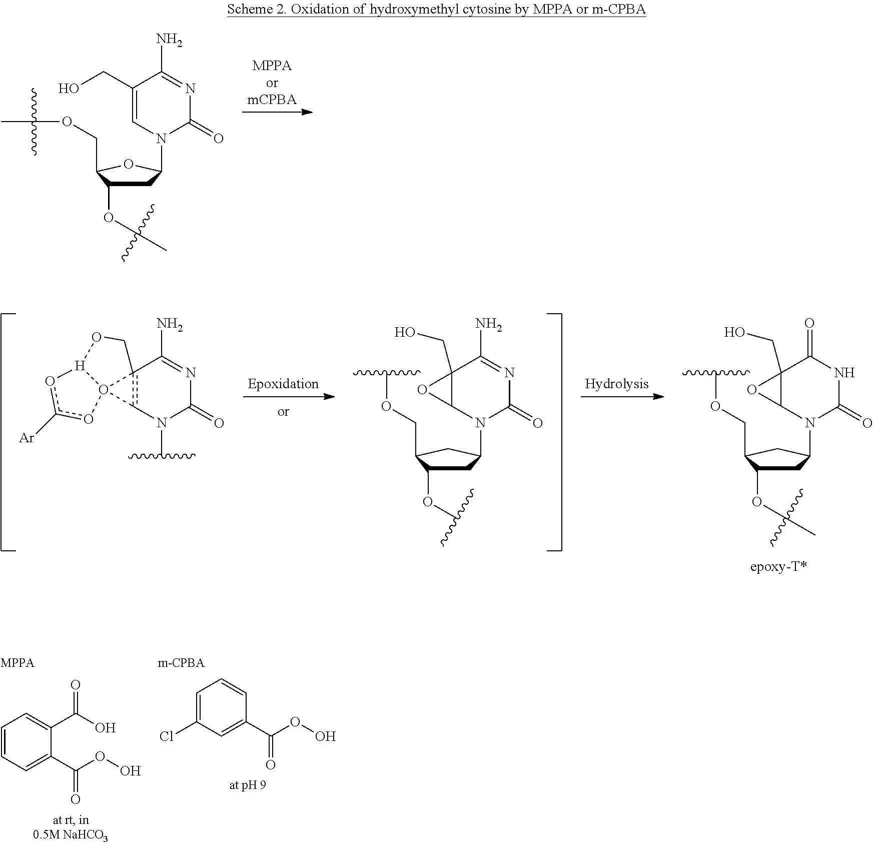

In some other embodiments, the oxidative reagent may comprise hydrogen peroxide and one or more metal compounds, such as transition metal compounds. The transition metal compound may be selected from the group consisting of a molybdium derivative, a vanadium derivative, a tungsten derivative, and a rhenium derivative, and combinations thereof. The transition metal compounds could be used either in stoichiometric version or in a catalytic version in presence of hydrogen peroxide H₂O₂ and may perform dihydroxylation and/or epoxidation as illustrated in Scheme 3. Non-liming examples of molybdium derivatives includes molybdic acid, phosphomolybdic acid hydrate, bis(acetylacetonato)dioxomolybdenum(VI), molybdenum(VI) dichloride dioxide, molybdenum(II) acetate dimer, and combinations thereof. Non-limiting examples of vanadium derivatives include vanadium(IV) oxide sulfate hydrate, vanadium(IV) oxide, or a combination thereof. Non-limiting tungsten derivatives include tungstic acid, tungsten(VI) dichloride dioxide, tungsten(VI) oxychloride, or combinations thereof. Non-limiting examples of or rhenium derivatives include methyltrioxorhenium (VII), rhenium(VII) oxide, or a combination thereof.

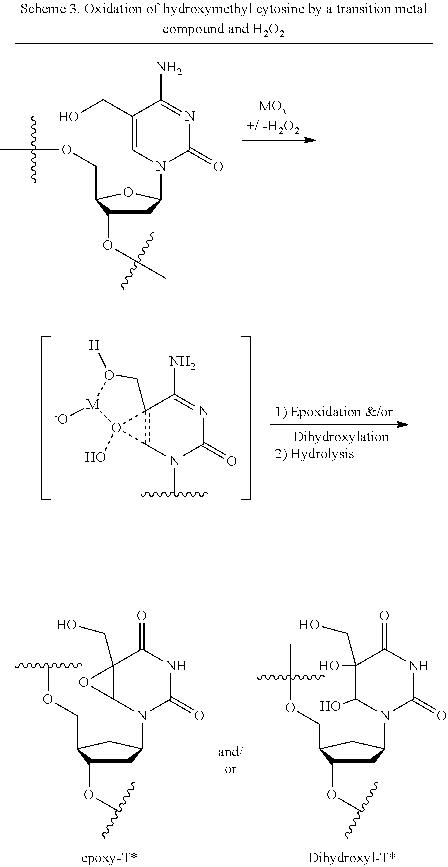

The oxidation method described herein may also be used to determine or identify cytosine methylation of a nucleic acid sequence in a nucleic acid sample by identifying both methylated cytosines (mC) and hydroxymethylated cytosines (hmC). The method may comprise:
contacting the nucleic acid sample with a TET enzyme to convert methylated cytosines to hydroxymethylated cytosines in the nucleic acid sequence;
reacting hydroxymethylated cytosines in the TET treated nucleic acid sample with a composition comprising an oxidative reagent to convert hydroxymethylated cytosines to modified thymine moieties each having the structure of Formula (I) or (II):

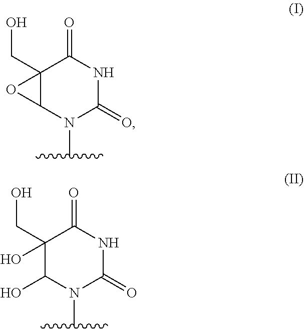

to form a modified nucleic acid sequence; and
amplifying the modified nucleic acid sequence. This method involves the use of a TET example, which readily converts mC to hmC. In some such embodiment of the method, the oxidative reagents used for converting hydroxymethylated cytosines to the modified thymine moieties may be the same as those described above.

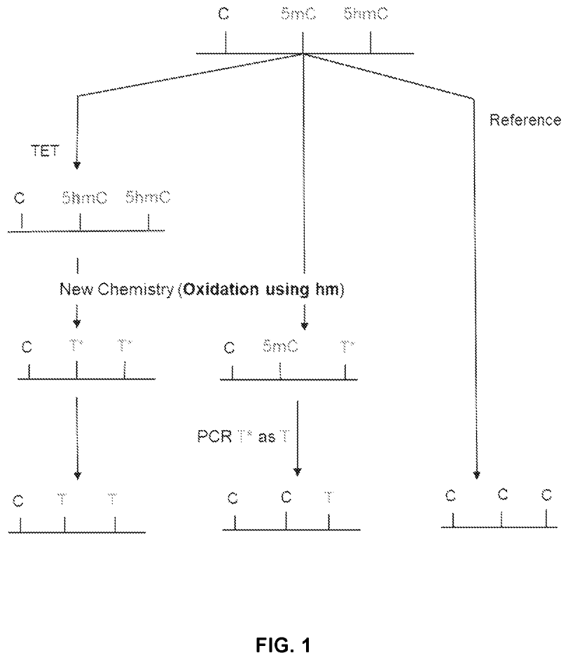

In any embodiments of the oxidative method described herein, the method may further include sequencing the amplified modified nucleic acid sequence; and determining the sites of the modified thymine moieties by comparing the modified nucleic acid sequence to a reference unconverted nucleic acid sequence. In some such embodiment, the sequencing method used may be sequencing by synthesis (SBS). The oxidative method described herein for detecting mC and hmC is further illustrated in FIG. 1.

### Method of Methylation Detection by Forming Pseudo Thymine-Like Imino Tautomers

Another aspect of the present disclosure relates to a method of identifying one or more hydroxymethylated cytosines of a nucleic acid sequence in a nucleic acid sample, comprising:
contacting the nucleic acid sample with

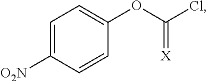

wherein X is O or S;
converting the hydroxymethylated cytosines to pseudo thymine moieties each having the structure of Formula (IIIa) or (IIIb):

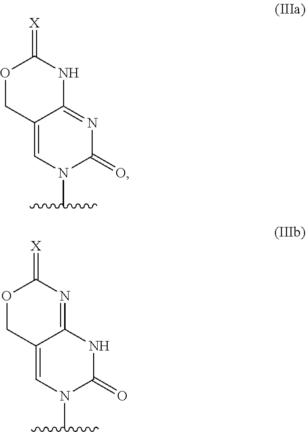

to form a modified nucleic acid sequence; and
amplifying the modified nucleic acid sequence.

A further aspect of the present disclosure relates to a method of identifying one or more hydroxymethylated cytosines of a nucleic acid sequence in a nucleic acid sample, comprising:
contacting the nucleic acid sample with

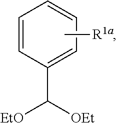

wherein R^{1a} is an optionally present hydrophilic electron withdrawing group;
converting the hydroxymethylated cytosines to pseudo thymine moieties having the structure of Formula (IVb):

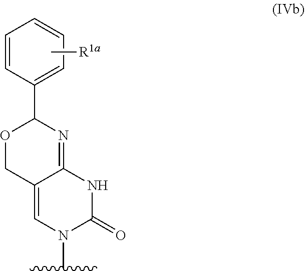

to form a modified nucleic acid sequence; and amplifying the modified nucleic acid sequence. In some embodiments, R^{1a} is at the para and/or ortho position. In further embodiments, R^{1a} may be sulfonate (—SO₃⁻) or a primary sulfonamide (—SO₂NH₂).

Both methods rely on the chemical modification of hydroxymethyl cytosine to form one or more imino tautomers which may be recognized as a pseudo thymine, which is illustrated in Schemes 4a and 4b below. The mC or hmC is attached to a 2-deoxyribose ring of the nucleoside or nucleotide, which may be part of an oligonucleotide, a polynucleotide, or a nucleic acid sequence.

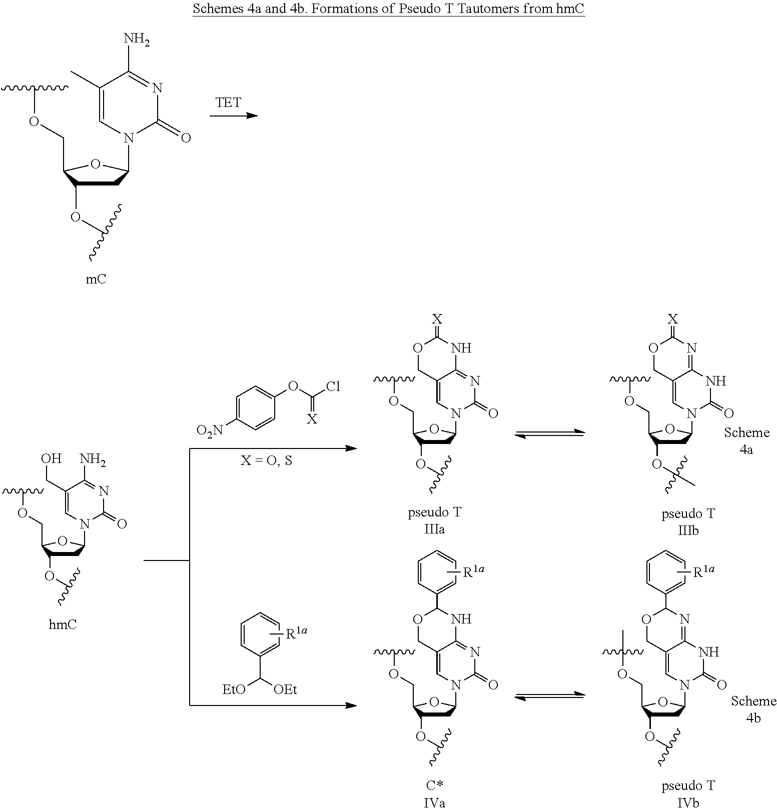

In Scheme 4a, mc is first converted to hmC by TET, then reacted with

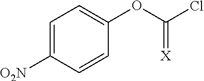

to form two tautomers of formula (IIIa) and (IIIb), and either tautomer may be the main form. Because of the extra electron acceptor is introduced, compound of Formula (IIIa) may act as both as a modified cytosine and a pseudo thymine. In Scheme 4b, hmC reacts with

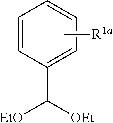

to form tautomers of Formula (IVa) and (IVb), and either tautomer may be the main form. Tautomer IVa is the modified cytosine and Tautomer IVb is the pseudo T form.

Furthermore, both methods may also be used to determine or identify cytosine methylation of a nucleic acid sequence in a nucleic acid sample by identifying both methylated cytosines (mC) and hydroxymethylated cytosines (hmC). The method may comprise:
contacting the nucleic acid sample with a TET enzyme to convert methylated cytosines to hydroxymethylated cytosines in the nucleic acid sequence;
reacting hydroxymethylated cytosines in the TET treated nucleic acid sample with

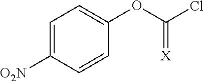

to convert hydroxymethylated cytosines to pseudo thymine moieties each having the structure of Formula (IIIa) or (IIIb):

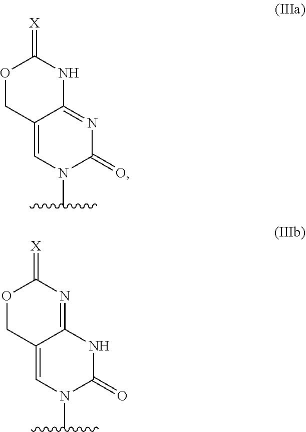

to form a modified nucleic acid sequence, wherein X is O or S; and amplifying the modified nucleic acid sequence.

Alternatively, the method may comprise: contacting the nucleic acid sample with a TET enzyme to convert methylated cytosines to hydroxymethylated cytosines in the nucleic acid sequence; reacting hydroxymethylated cytosines in the TET treated nucleic acid sample with

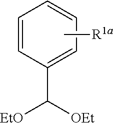

to convert hydroxymethylated cytosines to pseudo thymine moieties each having the structure of Formula (IVb):

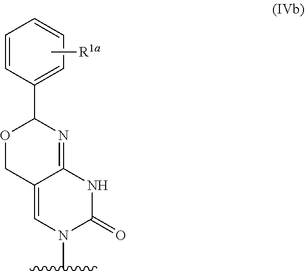

to form a modified nucleic acid sequence, wherein R^{1a} is an optionally present hydrophilic electron withdrawing group described herein; and amplifying the modified nucleic acid sequence.

There is concern that the treatment of mC with TET might not stop at hmC stage, instead going further to fC or caC. An additional aspect of the imino tautomer method described herein involves the conversion of hmC to 5-carboxylated cytosine (caC or 5-caC), then a similar modification to facilitate the conversion of cytosine to pseudo-T imino tautomer.

For example, the method may comprise: contacting the nucleic acid sample with a TET enzyme to convert methylated and hydroxymethylated cytosines in the nucleic acid sequence to carboxylated cytosines; reacting carboxylated cytosines in the TET treated nucleic acid sample with a cyanate or thiocyanate to convert carboxylated cytosines to pseudo thymine moieties each having the structure of Formula (IIId):

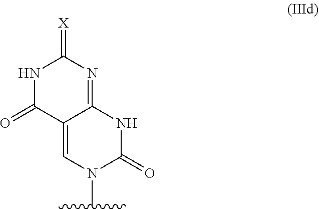

to form a modified nucleic acid sequence, wherein X is O or S; and amplifying the modified nucleic acid sequence. In some embodiments, X is O. In some embodiments, the cyanate reagent is an inorganic cyanate salt, such as potassium cyanate (KOCN) or sodium cyanate (NaOCN).

Alternatively, the method may comprise: contacting the nucleic acid sample with a TET enzyme to convert methylated and hydroxymethylated cytosines in the nucleic acid sequence to carboxylated cytosines; reacting carboxylated cytosines in the TET treated nucleic acid sample first with ammonia in the presence of a carboxyl activating agent, then reacting with

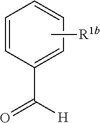

to convert carboxylated cytosines to pseudo thymine moieties each having the structure of Formula (IVd):

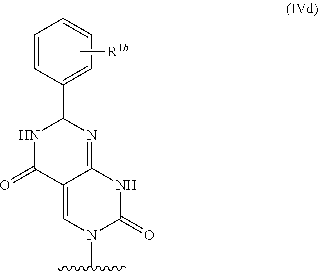

to form a modified nucleic acid sequence, wherein R^{1b} is an optionally present hydrophilic group; and amplifying the modified nucleic acid sequence. In some embodiments, R^{1b} may be at the para or ortho position. In further embodiments, R^{1b} may be —SO₃⁻ or —SO₂NH₂. In some embodiments, the carboxyl activating agent is DCC or EDC.

The TET facilitated caC conversion and subsequent imino tautomer formations are further illustrated in Schemes 5a and 5b below. The mC or hmC is attached to a 2-deoxyribose ring of the nucleoside or nucleotide, which may be part of an oligonucleotide, a polynucleotide, or a nucleic acid sequence.

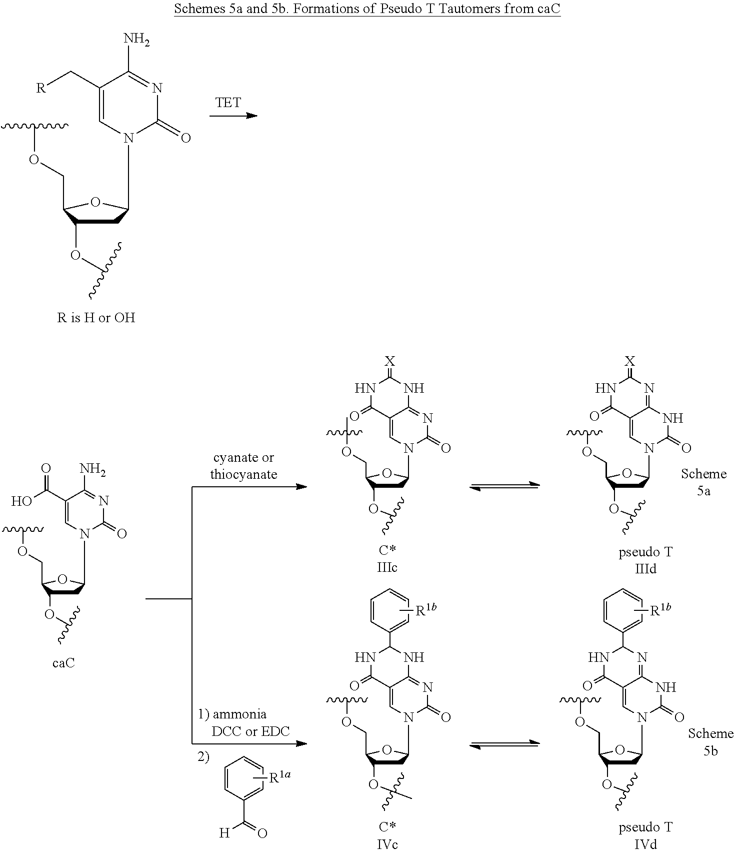

In Scheme 5a, mC is first converted to hmC by TET, then both mC and hmC are further converted by TET to the final oxidation product caC, which then reacted with cyanate R′OCN (X═O) or thiocyanate R′SCN (X═S) to form two tautomers of formula (IIIc) and (IIId), and either tautomer may be the main form. Tautomer of Formula (IIId) may act as a pseudo thymine. In Scheme 5b, caC first reacts with ammonia in the presence of a carboxyl activating agent such as DCC or EDC to convert the carboxyl group to amide, then the intermediate amide reacts with

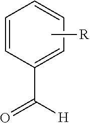

to form tautomers of Formula (IVc) and (IVd) and either tautomer may be the main form. Tautomer IVc is the modified cytosine and Tautomer IVd is the pseudo-T form. Alternatively, caC may direct react with an optionally substituted benzonitrile to arrive at tautomers of IVc and IVd.

In any embodiments of the imino tautomer pseudo-T conversion methods described herein, the method further comprises: sequencing the amplified modified nucleic acid sequence; and determining the sites of pseudo thymine moieties by comparing the modified nucleic acid sequence to a reference nucleic acid sequence. In some such embodiment, the sequencing method used may be SBS. The oxidative method described herein for detecting mC and hmC is further illustrated in FIG. 1.

### Method of Methylation Detection by Michael Addition or Cycloaddition

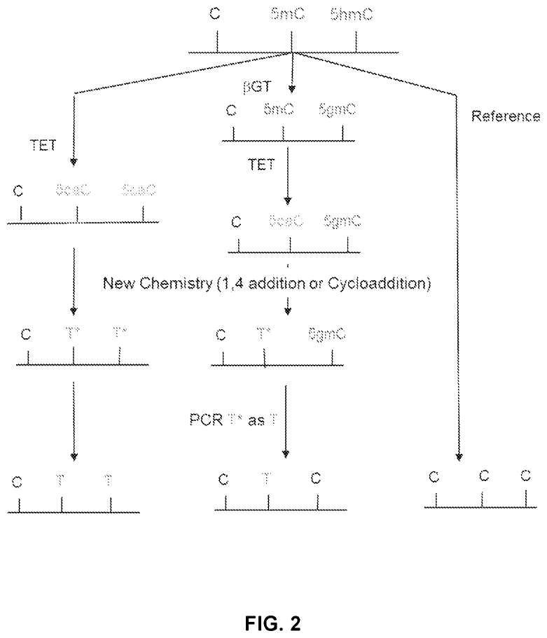

Additional methods described here use Michael Addition (e.g., 1,4-Michael Addition) or cycloaddition (e.g., Diels Alder [4+2] cycloaddition) in combination with TET enzymology and β-glucosyltransferase (β-GT) to convert selectively 5mC and/or 5hmC into a T equivalent (U, bicyclic T, other modified T* or U*) through caC (FIG. 2). The chemistries leverage the electron-withdrawing character of the carboxy group in caC. This is activating the adjacent double bond offering an adequate site for a Michael 1,4-Addition or a cycloaddition (Scheme 6). Resulted product will undergo hydrolysis resulting at the conversion to pseudo-T (T*) or U. As depicted in Scheme 6, the 5caC is attached to a 2-deoxyribose ring of the nucleoside or nucleotide, which may be part of an oligonucleotide, a polynucleotide, or a nucleic acid sequence.

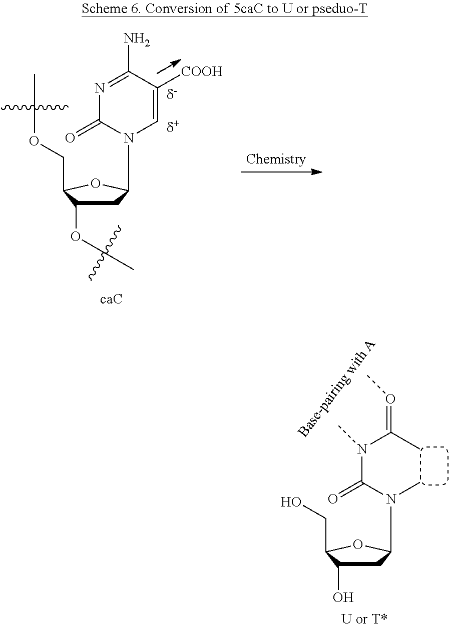

In some embodiments, the Michael Addition chemistry maybe used in a method of identifying methylated and hydroxymethylated cytosines of a nucleic acid sequence in a nucleic acid sample, comprising:
contacting the nucleic acid sample with a TET enzyme to convert methylated and hydroxymethylated cytosines in the nucleic acid sequence to carboxylated cytosines;
reacting carboxylated cytosines in the TET treated nucleic acid sample with

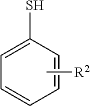

in a Michael Addition reaction to convert carboxylated cytosines to first intermediates each having the structure of Formula (Va):

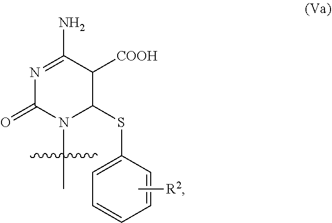

wherein R² is 4-OCH₃, 4-CH₃, 2-OCH₃, 4-Cl, 4-NO₂, or 4-CF₃;

treating the first intermediates with hydrogen peroxide to form second intermediates having the structure of Formula (Vb):

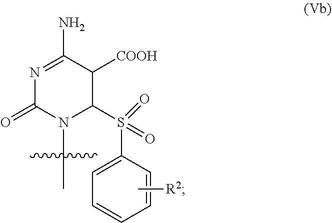

reacting the second intermediate with 1,8-diazabicyclo[5.4.0]undec-7-ene (DBU) to convert the second intermediate to a uracil moiety to form a modified nucleic acid sequence; and amplifying the modified nucleic acid sequence.

For Michael 1,4-Addition, a variety of nucleophiles can be used. As an example, the addition of thiophenol is depicted in Scheme 7. The mC or hmC is attached to a 2-deoxyribose ring of the nucleoside or nucleotide, which may be part of an oligonucleotide, a polynucleotide, or a nucleic acid sequence. First, both mC and hmC are converted to caC by TET. Then, caC reacts with an aryl thiol compound

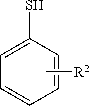

to convert caC to a first intermediate C* of formula (Va)

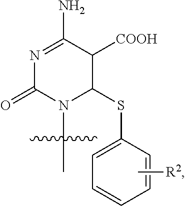

in which the aromatic system of nucleobase is broken. Subsequent oxidation with H₂O₂ and hydrolysis give to a second intermediate U* of formula (Vb), which may then be converted to U in basic conditions in the presence of DBU.

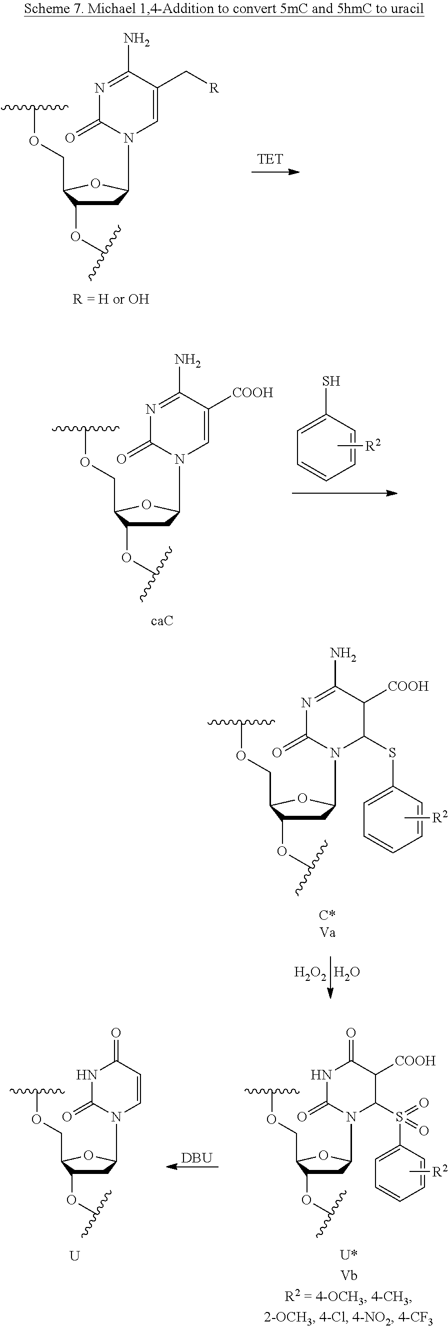

This method may also be used in selective identification of 5mC, which utilizes β-GT to label 5hmC with glucose and thereby protect it from TET oxidation. In this method, TET only converts 5mC to 5caC, therefore may be used in the identification of methylated cytosines of a nucleic acid sequence in a nucleic acid sample. In such embodiment, the method comprises:
contacting the nucleic acid sample with β-GT to selectively glucosylating hydroxymethyl cytosines of the nucleic acid sequence;
contacting the β-GT treated nucleic acid sample with a TET enzyme to convert methylated cytosines in the nucleic acid sequence to carboxylated cytosines;
reacting carboxylated cytosines in the TET treated nucleic acid sample with

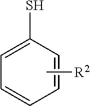

in a Michael Addition reaction to convert carboxylated cytosines to first intermediates each having the structure of Formula (Va):

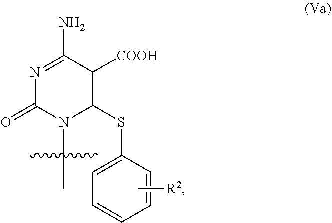

wherein R² is 4-OCH₃, 4-CH₃, 2-OCH₃, 4-Cl, 4-NO₂, or 4-CF₃;
treating the first intermediates with hydrogen peroxide to form second intermediates each having the structure of Formula (Vb):

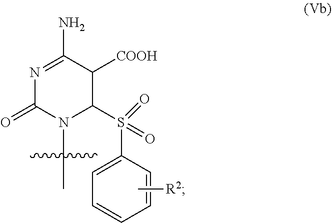

reacting the second intermediates with DBU to convert the second intermediates to uracil moieties to form a modified nucleic acid sequence; and amplifying the modified nucleic acid sequence.

In some embodiments of the Michael Addition method described herein, the method further comprises: sequencing the amplified modified nucleic acid sequence; and determining the sites of converted uracil moieties by comparing the modified nucleic acid sequence to a reference nucleic acid sequence. In some such embodiment, the sequencing method used may be SBS.

Similarly, leveraging the specific properties of caC, cycloadditions could be used to form a bicyclic T moiety (T*) through cycloaddition reaction. A further aspect of the present application relates to a method of identifying cytosine methylation of a nucleic acid sequence in a nucleic acid sample, comprising:
contacting the nucleic acid sample with a TET enzyme to convert methylated and hydroxymethylated cytosines in the nucleic acid sequence to carboxylated cytosines;
reacting the TET treated nucleic acid sample with an unsaturated reagent in a cycloaddition reaction to convert carboxylated cytosines to first intermediates each having the structure of Formula (VI):

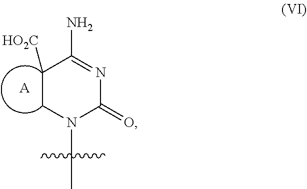

wherein ring A is an optionally substituted 4, 5 or 6 membered carbocyclyl or heterocyclyl ring;
converting the first intermediates to bicyclic thymine moieties each having a structure of Formula (VII):

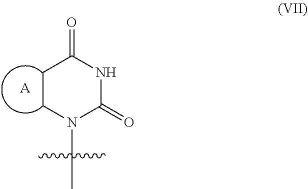

to form a modified nucleic acid sequence; and amplifying the modified nucleic acid sequence.

As depicted in Scheme 8, the mC or hmC is attached to a 2-deoxyribose ring of the nucleoside or nucleotide, which may be part of an oligonucleotide, a polynucleotide, or a nucleic acid sequence.

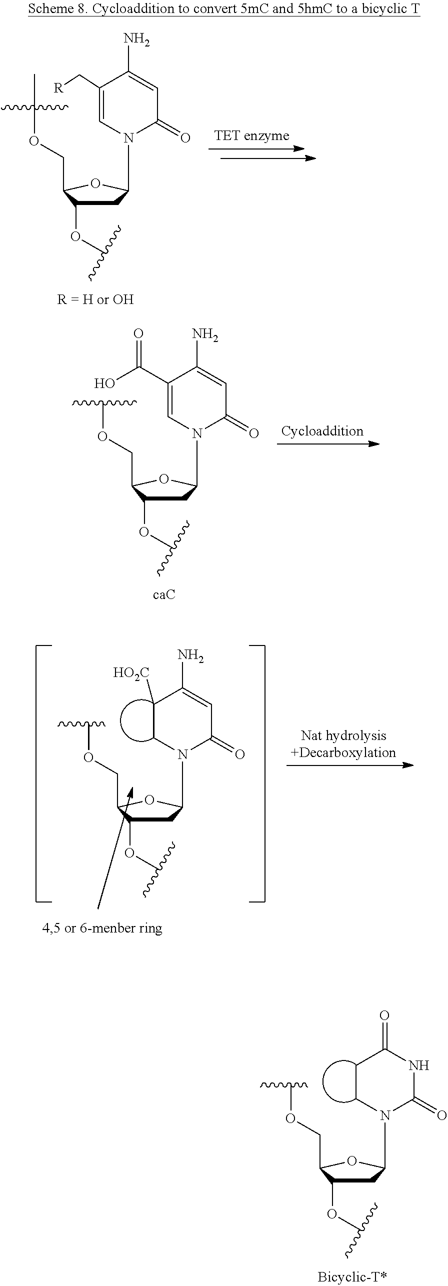

Similarly, this method may also be used in selective identification of 5mC, which utilizes β-GT to label 5hmC with glucose and thereby protect it from TET oxidation. In this method, TET only converts 5mC to 5caC, therefore may be used in the identification of methylated cytosines of a nucleic acid sequence in a nucleic acid sample. In such embodiment, the method comprises:
contacting the nucleic acid sample with β-glucosyltransferase (β-GT) to selectively glucosylating hydroxymethyl cytosines of the nucleic acid sequence;
contacting the β-GT treated nucleic acid sample with a TET enzyme to convert methylated cytosines in the nucleic acid sequence to carboxylated cytosines;
reacting carboxylated cytosines in the TET treated nucleic acid sample with an unsaturated reagent in a cycloaddition reaction to convert carboxylated cytosines to first intermediates each having the structure of Formula (VI):

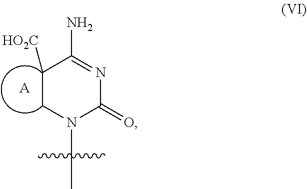

wherein ring A is an optionally substituted 4, 5 or 6 membered carbocyclyl or heterocyclyl ring;
converting the first intermediates to bicyclic thymine moieties each having a structure of Formula (VII):

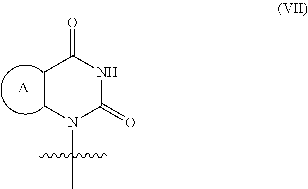

to form a modified nucleic acid sequence; and amplifying the modified nucleic acid sequence.

In some embodiments of the cycloaddition methods described herein, the unsaturated reagent is a 1,4-diene (for example

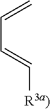

for example, and the bicyclic thymine moiety having a structure of Formula (VIIa):

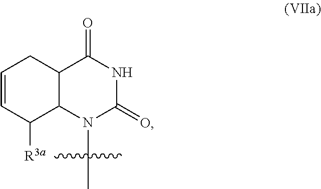

wherein R^{3a} is C₁-C₆ alkyl group optionally substituted with one or more hydrophilic moieties. In further embodiments, R^{3a} is C₁-C₆ alkyl substituted with one or more of —SO₃⁻ or —SO₂NH₂. In further embodiments, the 1,4-diene described herein may be further substituted, for example,

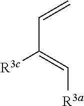

where R^{3c} is an electron donating group (e.g., C₁-C₆ alkoxy, —OSiR₃, —NR₂, —SiR₃, or a hydrophilic donating aromatic group, and R may be H or optionally substituted C₁-C₆ alkyl). In other embodiments, the unsaturated reagent is an azide (for example, R^{3b}—CH₂—N₃) and the bicyclic thymine moiety having a structure of Formula (VIIb):

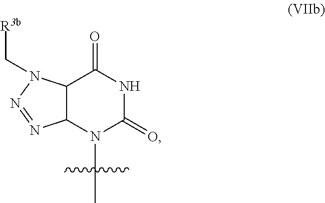

wherein R^{3b} is C₁-C₆ alkyl group optionally substituted with one or more hydrophilic moieties. In further embodiments, R^{3b} is C₁-C₆ alkyl substituted with one or more of —SO₃⁻ or —SO₂NH₂. More specifically and as a non-limiting example, Diels-Alder or “ene”-Click cycloadditions could be used as depicted in Scheme 9.

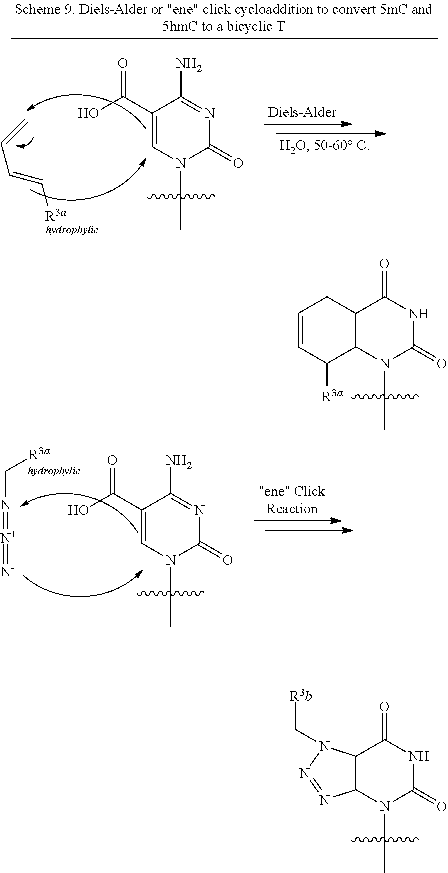

In some embodiments, the cycloaddition method further comprises: sequencing the amplified modified nucleic acid sequence; and determining the sites of bicyclic thymine moieties by comparing the modified nucleic acid sequence to a reference nucleic acid sequence. In some such embodiment, the sequencing method used may be SBS.

In any embodiments of the methods described herein, the nucleic acid sample is a genomic DNA sample. In further embodiment, the sample may be a cell-free DNA sample.

In any reaction schemes described herein where mC, hmC or caC is attached to a 2-deoxyribose ring of the nucleoside or nucleotide, it is also contemplated that the mC, hmC or caC may be attached to a ribose ring of the nucleoside or nucleotide (e.g., a RNA sample), or any non-natural or modified sugar moieties of the nucleoside/nucleotide.

### Methods of Sequencing

Some embodiments are directed to methods of detecting the sites of converted mC or hmC in an oligonucleotide, polynucleotide, or a nucleic acid sequence, using one of the methods described herein. In one embodiment, the detecting includes determining a nucleotide sequence of the oligonucleotide, polynucleotide, or the nucleic acid using any one of the sequencing methods described herein. In one particular example, the sequencing method is SBS.

Some embodiments that use nucleic acids can include a step of amplifying the nucleic acids on the substrate. Many different DNA amplification techniques can be used in conjunction with the substrates described herein. Exemplary techniques that can be used include, but are not limited to, polymerase chain reaction (PCR), rolling circle amplification (RCA), multiple displacement amplification (MDA), or random prime amplification (RPA). In particular embodiments, one or more oligonucleotide primers used for amplification can be attached to a substrate (e.g., via the azido silane layer). In PCR embodiments, one or both of the primers used for amplification can be attached to the substrate. Formats that utilize two species of attached primer are often referred to as bridge amplification because double stranded amplicons form a bridge-like structure between the two attached primers that flank the template sequence that has been copied. Exemplary reagents and conditions that can be used for bridge amplification are described, for example, in U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2004/0002090; U.S. Patent Publ. No 2007/0128624; and U.S. Patent Publ. No. 2008/0009420, each of which is incorporated herein by reference.

PCR amplification can also be carried out with one amplification primer attached to a substrate and a second primer in solution. An exemplary format that uses a combination of one attached primer and soluble primer is emulsion PCR as described, for example, in Dressman et al., Proc. Natl. Acad. Sci. USA 100:8817-8822 (2003), WO 05/010145, or U.S. Patent Publ. Nos. 2005/0130173 or 2005/0064460, each of which is incorporated herein by reference. Emulsion PCR is illustrative of the format and it will be understood that for purposes of the methods set forth herein the use of an emulsion is optional and indeed for several embodiments an emulsion is not used. Furthermore, primers need not be attached directly to substrate or solid supports as set forth in the ePCR references and can instead be attached to a gel or polymer coating as set forth herein.

RCA techniques can be modified for use in a method of the present disclosure. Exemplary components that can be used in an RCA reaction and principles by which RCA produces amplicons are described, for example, in Lizardi et al., Nat. Genet. 19:225-232 (1998) and US 2007/0099208 A1, each of which is incorporated herein by reference. Primers used for RCA can be in solution or attached to a gel or polymer coating.

MIDA techniques can be modified for use in a method of the present disclosure. Some basic principles and useful conditions for MDA are described, for example, in Dean et al., Proc Natl. Acad. Sci. USA 99:5261-66 (2002); Lage et al., Genome Research 13:294-307 (2003); Walker et al., Molecular Methods for Virus Detection, Academic Press, Inc., 1995; Walker et al., Nucl. Acids Res. 20:1691-96 (1992); U.S. Pat. Nos. 5,455,166; 5,130,238; and 6,214,587, each of which is incorporated herein by reference. Primers used for MDA can be in solution or attached to a gel or polymer coating.

In particular embodiments a combination of the above-exemplified amplification techniques can be used. For example, RCA and MDA can be used in a combination wherein RCA is used to generate a concatameric amplicon in solution (e.g., using solution-phase primers). The amplicon can then be used as a template for MDA using primers that are attached to a substrate (e.g., via a gel or polymer coating). In this example, amplicons produced after the combined RCA and MDA steps will be attached to the substrate.

Substrates of the present disclosure that contain nucleic acid arrays can be used for any of a variety of purposes. A particularly desirable use for the nucleic acids is to serve as capture probes that hybridize to target nucleic acids having complementary sequences. The target nucleic acids once hybridized to the capture probes can be detected, for example, via a label recruited to the capture probe. Methods for detection of target nucleic acids via hybridization to capture probes are known in the art and include, for example, those described in U.S. Pat. Nos. 7,582,420, 6,890,741; 6,913,884 or 6,355,431 or U.S. Pat. Pub Nos. 2005/0053980 A1, 2009/0186349 A1 or 2005/0181440 A1, each of which is incorporated herein by reference. For example, a label can be recruited to a capture probe by virtue of hybridization of the capture probe to a target probe that bears the label. In another example, a label can be recruited to a capture probe by hybridizing a target probe to the capture probe such that the capture probe can be extended by ligation to a labeled oligonucleotide (e.g., via ligase activity) or by addition of a labeled nucleotide (e.g., via polymerase activity).

In some embodiments, a substrate described herein can be used for determining a nucleotide sequence of a polynucleotide. In such embodiments, the method can comprise the steps of (a) contacting a substrate-attached polynucleotide/copy polynucleotide complex with one or more different type of nucleotides in the presence of a polymerase (e.g., DNA polymerase); (b) incorporating one type of nucleotide to the copy polynucleotide strand to form an extended copy polynucleotide; (c) perform one or more fluorescent measurements of one or more the extended copy polynucleotides; wherein steps (a) to (c) are repeated, thereby determining the sequence of the substrate-attached polynucleotide.

Nucleic acid sequencing can be used to determine a nucleotide sequence of a polynucleotide by various processes known in the art. In a preferred method, sequencing-by-synthesis (SBS) is utilized to determine a nucleotide sequence of a polynucleotide attached to a surface of a substrate (e.g., via any one of the polymer coatings described herein). In such a process, one or more nucleotides are provided to a template polynucleotide that is associated with a polynucleotide polymerase. The polynucleotide polymerase incorporates the one or more nucleotides into a newly synthesized nucleic acid strand that is complementary to the polynucleotide template. The synthesis is initiated from an oligonucleotide primer that is complementary to a portion of the template polynucleotide or to a portion of a universal or non-variable nucleic acid that is covalently bound at one end of the template polynucleotide. As nucleotides are incorporated against the template polynucleotide, a detectable signal is generated that allows for the determination of which nucleotide has been incorporated during each step of the sequencing process. In this way, the sequence of a nucleic acid complementary to at least a portion of the template polynucleotide can be generated, thereby permitting determination of the nucleotide sequence of at least a portion of the template polynucleotide.

Flow cells provide a convenient format for housing an array that is produced by the methods of the present disclosure and that is subjected to a sequencing-by-synthesis (SBS) or other detection technique that involves repeated delivery of reagents in cycles. For example, to initiate a first SBS cycle, one or more labeled nucleotides, DNA polymerase, etc., can be flowed into/through a flow cell that houses a nucleic acid array made by methods set forth herein. Those sites of an array where primer extension causes a labeled nucleotide to be incorporated can be detected. Optionally, the nucleotides can further include a reversible termination property that terminates further primer extension once a nucleotide has been added to a primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with an array produced by the methods of the present disclosure are described, for example, in Bentley et al., Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference in its entirety.

In some embodiments of the above-described method, which employ a flow cell, only a single type of nucleotide is present in the flow cell during a single flow step. In such embodiments, the nucleotide can be selected from the group consisting of dATP, dCTP, dGTP, dTTP, and analogs thereof. In other embodiments of the above-described method which employ a flow cell, a plurality different types of nucleotides are present in the flow cell during a single flow step. In such methods, the nucleotides can be selected from dATP, dCTP, dGTP, dTTP, and analogs thereof.

Determination of the nucleotide or nucleotides incorporated during each flow step for one or more of the polynucleotides attached to the polymer coating on the surface of the substrate present in the flow cell is achieved by detecting a signal produced at or near the polynucleotide template. In some embodiments of the above-described methods, the detectable signal comprises an optical signal. In other embodiments, the detectable signal comprises a non-optical signal. In such embodiments, the non-optical signal comprises a change in pH at or near one or more of the polynucleotide templates.

Applications and uses of substrates of the present disclosure have been exemplified herein with regard to nucleic acids. However, it will be understood that other analytes can be attached to a substrate set forth herein and analyzed. One or more analytes can be present in or on a substrate of the present disclosure. The substrates of the present disclosure are particularly useful for detection of analytes, or for carrying out synthetic reactions with analytes. Thus, any of a variety of analytes that are to be detected, characterized, modified, synthesized, or the like can be present in or on a substrate set forth herein. Exemplary analytes include, but are not limited to, nucleic acids (e.g., DNA, RNA or analogs thereof), proteins, polysaccharides, cells, antibodies, epitopes, receptors, ligands, enzymes (e.g., kinases, phosphatases or polymerases), small molecule drug candidates, or the like. A substrate can include multiple different species from a library of analytes. For example, the species can be different antibodies from an antibody library, nucleic acids having different sequences from a library of nucleic acids, proteins having different structure and/or function from a library of proteins, drug candidates from a combinatorial library of small molecules, etc.

In some embodiments, analytes can be distributed to features on a substrate such that they are individually resolvable. For example, a single molecule of each analyte can be present at each feature. Alternatively, analytes can be present as colonies or populations such that individual molecules are not necessarily resolved. The colonies or populations can be homogenous with respect to containing only a single species of analyte (albeit in multiple copies). Taking nucleic acids as an example, each feature on a substrate can include a colony or population of nucleic acids and every nucleic acid in the colony or population can have the same nucleotide sequence (either single stranded or double stranded). Such colonies can be created by cluster amplification or bridge amplification as set forth previously herein. Multiple repeats of a target sequence can be present in a single nucleic acid molecule, such as a concatamer created using a rolling circle amplification procedure. Thus, a feature on a substrate can contain multiple copies of a single species of an analyte. Alternatively, a colony or population of analytes that are at a feature can include two or more different species. For example, one or more wells on a substrate can each contain a mixed colony having two or more different nucleic acid species (i.e., nucleic acid molecules with different sequences). The two or more nucleic acid species in a mixed colony can be present in non-negligible amounts, for example, allowing more than one nucleic acid to be detected in the mixed colony.

In specific non-limiting embodiments, the disclosure encompasses methods of nucleic acid sequencing, re-sequencing, whole genome sequencing, single nucleotide polymorphism scoring, any other application involving the detection of the labeled nucleotide or nucleoside set forth herein when incorporated into a polynucleotide. Any of a variety of other applications benefitting the use of polynucleotides labeled with the nucleotides comprising fluorescent dyes can use labeled nucleotides or nucleosides with dyes set forth herein.

In a particular embodiment, the disclosure provides use of labeled nucleotides according to the disclosure in a polynucleotide sequencing-by-synthesis (SBS) reaction. Sequencing-by-synthesis generally involves sequential addition of one or more nucleotides or oligonucleotides to a growing polynucleotide chain in the 5′ to 3′ direction using a polymerase or ligase in order to form an extended polynucleotide chain complementary to the template nucleic acid to be sequenced. The identity of the base present in one or more of the added nucleotide(s) can be determined in a detection or “imaging” step. The identity of the added base may be determined after each nucleotide incorporation step. The sequence of the template may then be inferred using conventional Watson-Crick base-pairing rules. The use of the labeled nucleotides set forth herein for determination of the identity of a single base may be useful, for example, in the scoring of single nucleotide polymorphisms, and such single base extension reactions are within the scope of this disclosure.

In an embodiment of the present disclosure, the sequence of a template polynucleotide is determined by detecting the incorporation of one or more 3′ blocked nucleotides described herein into a nascent strand complementary to the template polynucleotide to be sequenced through the detection of fluorescent label(s) attached to the incorporated nucleotide(s). Sequencing of the template polynucleotide can be primed with a suitable primer (or prepared as a hairpin construct which will contain the primer as part of the hairpin), and the nascent chain is extended in a stepwise manner by addition of nucleotides to the 3′ end of the primer in a polymerase-catalyzed reaction.

In particular embodiments, each of the different nucleotide triphosphates (A, T, G and C) may be labeled with a unique fluorophore and also comprises a blocking group at the 3′ position to prevent uncontrolled polymerization. Alternatively, one of the four nucleotides may be unlabeled (dark). The polymerase enzyme incorporates a nucleotide into the nascent chain complementary to the template polynucleotide, and the blocking group prevents further incorporation of nucleotides. Any unincorporated nucleotides can be washed away and the fluorescent signal from each incorporated nucleotide can be “read” optically by suitable means. such as a charge-coupled device using laser excitation and suitable emission filters. The 3′-blocking group and fluorescent dye compounds can then be removed (deprotected) simultaneously or sequentially to expose the nascent chain for further nucleotide incorporation. Typically, the identity of the incorporated nucleotide will be determined after each incorporation step, but this is not strictly essential. Similarly, U.S. Pat. No. 5,302,509 (which is incorporated herein by reference) discloses a method to sequence polynucleotides immobilized on a solid support.

The method, as exemplified above, utilizes the incorporation of fluorescently labeled, 3′-blocked nucleotides A, G, C, and T into a growing strand complementary to the immobilized polynucleotide, in the presence of DNA polymerase. The polymerase incorporates a base complementary to the target polynucleotide but is prevented from further addition by the 3′-blocking group. The label of the incorporated nucleotide can then be determined, and the blocking group removed by chemical cleavage to allow further polymerization to occur. The nucleic acid template to be sequenced in a sequencing-by-synthesis reaction may be any polynucleotide that it is desired to sequence. The nucleic acid template for a sequencing reaction will typically comprise a double stranded region having a free 3′-OH group that serves as a primer or initiation point for the addition of further nucleotides in the sequencing reaction. The region of the template to be sequenced will overhang this free 3′-OH group on the complementary strand. The overhanging region of the template to be sequenced may be single stranded but can be double-stranded, provided that a “nick is present” on the strand complementary to the template strand to be sequenced to provide a free 3′-OH group for initiation of the sequencing reaction. In such embodiments, sequencing may proceed by strand displacement. In certain embodiments, a primer bearing the free 3′-OH group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Publication Nos. WO 01/57248 and WO 2005/047301, each of which is incorporated herein by reference. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′-OH group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

In certain embodiments, the nucleic acid template to be sequenced may be attached to a solid support via any suitable linkage method known in the art, for example via covalent attachment. In certain embodiments template polynucleotides may be attached directly to a solid support (e.g., a silica-based support). However, in other embodiments of the disclosure the surface of the solid support may be modified in some way so as to allow either direct covalent attachment of template polynucleotides, or to immobilize the template polynucleotides through a hydrogel or polyelectrolyte multilayer, which may itself be non-covalently attached to the solid support.

Some other embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B, Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing” Trends Biotechnol. 18, 147-151 (2000); Deamer, D. and D. Branton, “Characterization of nucleic acids by nanopore analysis”, Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007), Cockroft, S. L., Chu, J., Amorin, M. & Ghadiri, M. R. “A single-molecule nanopore device detects DNA polymerase activity with single-nucleotide resolution.”. J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some other embodiments of sequencing method involve nanoball sequencing technique, such as those described in U.S. Pat. No. 9,222,132, the disclosure of which is incorporated by reference. Through the process of rolling circle amplification (RCA), a large number of discrete DNA nanoballs may be generated. The nanoball mixture is then distributed onto a patterned slide surface containing features that allow a single nanoball to associate with each location. In DNA nanoball generation. DNA is fragmented and ligated to the first of four adapter sequences. The template is amplified, circularized and cleaved with a type II endonuclease. A second set of adapters is added, followed by amplification, circularization and cleavage. This process is repeated for the remaining two adapters. The final product is a circular template with four adapters, each separated by a template sequence. Library molecules undergo a rolling circle amplification step, generating a large mass of concatemers called DNA nanoballs, which are then deposited on a flow cell. Goodwin et al., “Coming of age: ten years of next-generation sequencing technologies,” Nat Rev Genet. 2016; 17(6):333-51.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414, both of which are incorporated herein by reference, or nucleotide incorporations can be detected with zero-mode waveguides as described, for example, in U.S. Pat. No. 7,315,019, which is incorporated herein by reference, and using fluorescent nucleotide analogs and engineered polymerases as described, for example, in U.S. Pat. No. 7,405,281 and U.S. Pub. No. 2008/0108082, both of which are incorporated herein by reference. The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Left. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

The present disclosure also encompasses dideoxynucleotides lacking hydroxyl groups at both of the 3′ and 2′ positions, such dideoxynucleotides being suitable for use in Sanger type sequencing methods and the like.

What is claimed is:

1. A method of identifying one or more hydroxymethylated cytosines of a nucleic acid sequence in a nucleic acid sample, comprising:
contacting the nucleic acid sample with a composition comprising an oxidative reagent;
converting the hydroxymethylated cytosines to modified thymine moieties each having the structure of Formula (I) or (II): 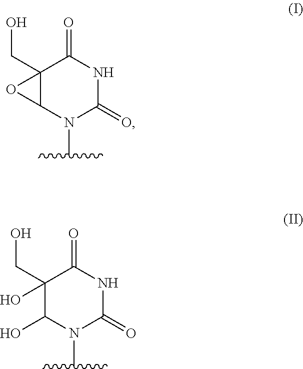 [SMILES: *N1C(=O)NC(=O)C2(CO)OC12 ; *N1C(=O)NC(=O)C(O)(CO)C1O]
to form a modified nucleic acid sequence; and
amplifying the modified nucleic acid sequence.

2. A method of identifying cytosine methylation of a nucleic acid sequence in a nucleic acid sample, comprising:
contacting the nucleic acid sample with a TET enzyme to convert one or more methylated cytosines to hydroxymethylated cytosines in the nucleic acid sequence;
reacting hydroxymethylated cytosines in the TET treated nucleic acid sample with a composition comprising an oxidative reagent to convert hydroxymethylated cytosines to modified thymine moieties each having the structure of Formula (I) or (II): 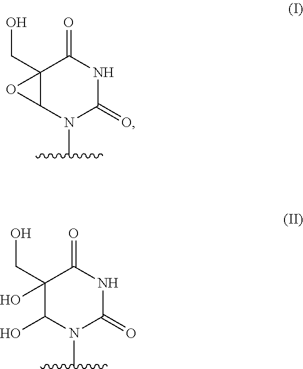 [SMILES: *N1C(=O)NC(=O)C2(CO)OC12 ; *N1C(=O)NC(=O)C(O)(CO)C1O]
to form a modified nucleic acid sequence; and
amplifying the modified nucleic acid sequence.

3. The method of claim 1 or 2, wherein the oxidative reagent reacts with hydroxymethylated cytosines to form epoxidation or dihydroxylation intermediates, and the method further comprises hydrolyzing the epoxidation or dihydroxylation intermediates to form the modified thymine moieties.

4. The method of any one of claims 1 to 3, further comprising:
sequencing the amplified modified nucleic acid sequence; and
determining the sites of modified thymine moieties by comparing the modified nucleic acid sequence to a reference nucleic acid sequence.

5. The method of any one of claims 1 to 4, wherein the oxidative reagent comprises a peracid.

6. The method of claim 5, wherein the peracid is 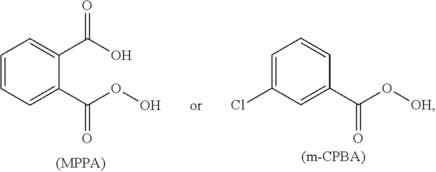 [SMILES: O=C(O)c1ccccc1C(=O)OO ; "or" ; O=C(OO)c1cccc(Cl)c1]
or a combination thereof.

7. The method of any one of claims 1 to 4, wherein the oxidative reagent comprises hydrogen peroxide and one or more transition metal compounds selected from the group consisting of a molybdium derivative, a vanadium derivative, a tungsten derivative, and a rhenium derivative, and combinations thereof.

8. The method of claim 7, wherein the molybdium derivative comprises molybdic acid, phosphomolybdic acid hydrate, bis(acetylacetonato)dioxomolybdenum(VI), molybdenum(VI) dichloride dioxide, molybdenum(II) acetate dimer, and combinations thereof.

9. The method of claim 7, wherein the vanadium derivative comprises vanadium(IV) oxide sulfate hydrate, vanadium(IV) oxide, and a combination thereof.

10. The method of claim 7, wherein the tungsten derivative comprises tungstic acid, tungsten(VI) dichloride dioxide, tungsten(VI) oxychloride, and combinations thereof.

11. The method of claim 7, wherein the rhenium derivative comprises methyltrioxorhenium (VII), rhenium(VII) oxide, and a combination thereof.

12. A method of identifying one or more hydroxymethylated cytosines of a nucleic acid sequence in a nucleic acid sample, comprising:
contacting the nucleic acid sample with 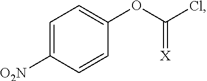
wherein X is O or S;
converting the hydroxymethylated cytosines to pseudo thymine moieties each having the structure of Formula (IIIa) or (IIIb): 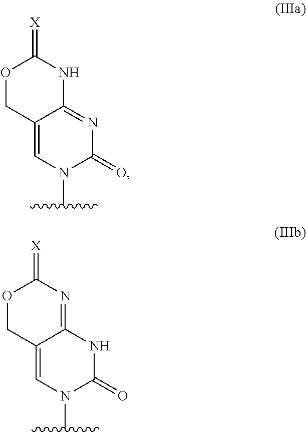
to form a modified nucleic acid sequence; and
amplifying the modified nucleic acid sequence.

13. A method of identifying cytosine methylation of a nucleic acid sequence in a nucleic acid sample, comprising:
contacting the nucleic acid sample with a TET enzyme to convert methylated cytosine to hydroxymethylated cytosines in the nucleic acid sequence;
reacting hydroxymethylated cytosines in the TET treated nucleic acid sample with 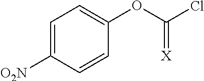
to convert hydroxymethylated cytosines to pseudo thymine moieties each having the structure of Formula (IIIa) or (IIIb): 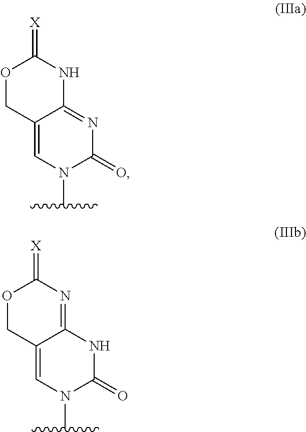
to form a modified nucleic acid sequence; and
amplifying the modified nucleic acid sequence;
wherein X is O or S.

14. A method of identifying cytosine methylation of a nucleic acid sequence in a nucleic acid sample, comprising:
contacting the nucleic acid sample with a TET enzyme to convert methylated and hydroxymethylated cytosines in the nucleic acid sequence to carboxylated cytosines;
reacting carboxylated cytosines in the TET treated nucleic acid sample with a cyanate or thiocyanate to convert carboxylated cytosines to pseudo thymine moieties each having the structure of Formula (IIId): 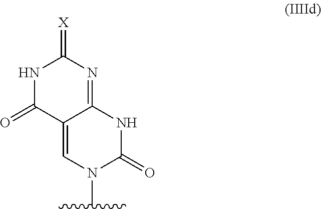
to form a modified nucleic acid sequence, wherein X is O or S; and
amplifying the modified nucleic acid sequence.

15. The method of any one of claims 12 to 14, wherein X is O.

16. A method of identifying one or more hydroxymethylated cytosines of a nucleic acid sequence in a nucleic acid sample, comprising:
contacting the nucleic acid sample with 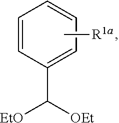
wherein R^{1a} is an optionally present hydrophilic electron withdrawing group;
converting the hydroxymethylated cytosines to pseudo thymine moieties having the structure of Formula (IVb): 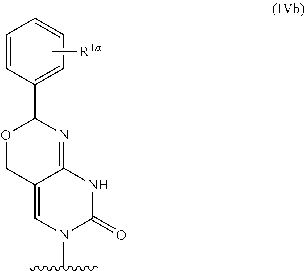
to form a modified nucleic acid sequence; and
amplifying the modified nucleic acid sequence.

17. A method of identifying cytosine methylation of a nucleic acid sequence in a nucleic acid sample, comprising:
contacting the nucleic acid sample with a TET enzyme to convert methylated cytosines to hydroxymethylated cytosines in the nucleic acid sequence;
reacting hydroxymethylated cytosines in the TET treated nucleic acid sample with 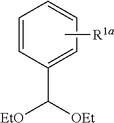
to convert hydroxymethylated cytosines to pseudo thymine moieties each having the structure of Formula (IVb): 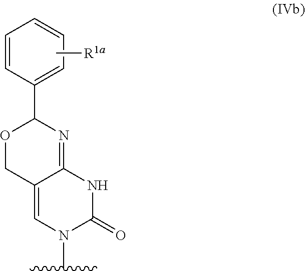
to form a modified nucleic acid sequence, wherein R^{1a} is an optionally present hydrophilic electron withdrawing group; and
amplifying the modified nucleic acid sequence.

18. A method of identifying cytosine methylation of a nucleic acid sequence in a nucleic acid sample, comprising:
contacting the nucleic acid sample with a TET enzyme to convert methylated cytosines and hydroxymethylated cytosines in the nucleic acid sequence to carboxylated cytosines;
reacting carboxylated cytosines in the TET treated nucleic acid sample first with ammonia in the presence of a carboxyl activating agent, then reacting with 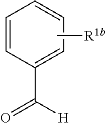
to convert carboxylated cytosines to pseudo thymine moieties each having the structure of Formula (IVd): 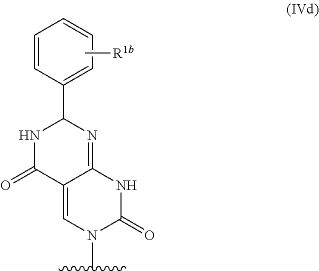
to form a modified nucleic acid sequence, wherein R^{1b} is an optionally present hydrophilic group; and
amplifying the modified nucleic acid sequence.

19. The method of any one of claims 12 to 18, further comprising:
sequencing the amplified modified nucleic acid sequence; and
determining the sites of pseudo thymine moieties by comparing the modified nucleic acid sequence to a reference nucleic acid sequence.

20. A method of identifying cytosine methylation of a nucleic acid sequence in a nucleic acid sample, comprising:
contacting the nucleic acid sample with a TET enzyme to convert methylated cytosines and hydroxymethylated cytosines in the nucleic acid sequence to carboxylated cytosines;
reacting carboxylated cytosines in the TET treated nucleic acid sample with 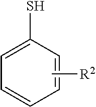
in a Michael Addition reaction to convert carboxylated cytosines to first intermediates each having the structure of Formula (Va): 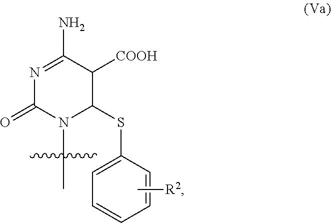
wherein R² is 4-OCH₃, 4-CH₃, 2-OCH₃, 4-Cl, 4-NO₂, or 4-CF₃;
treating the first intermediates with hydrogen peroxide to form second intermediates each having the structure of Formula (Vb): 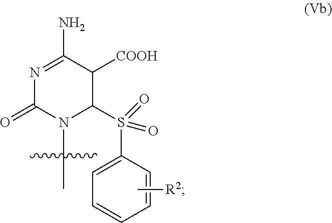
reacting the second intermediates with 1,8-diazabicyclo[5.4.0]undec-7-ene (DBU) to convert the second intermediates to uracil moieties to form a modified nucleic acid sequence; and
amplifying the modified nucleic acid sequence.

21. A method of identifying methylated cytosines of a nucleic acid sequence in a nucleic acid sample, comprising:
contacting the nucleic acid sample with β-glucosyltransferase (β-GT) to selectively glucosylating hydroxymethyl cytosines of the nucleic acid sequence;
contacting the β-GT treated nucleic acid sample with a TET enzyme to convert methylated cytosines in the nucleic acid sequence to carboxylated cytosines;
reacting carboxylated cytosines in the TET treated nucleic acid sample with 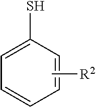
in a Michael Addition reaction to convert carboxylated cytosines to first intermediates each having the structure of Formula (Va): 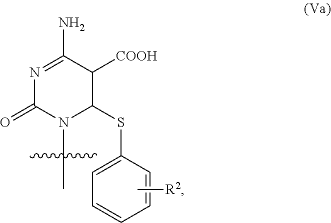
wherein R² is 4-OCH₃, 4-CH₃, 2-OCH₃, 4-Cl, 4-NO₂, or 4-CF₃;
treating the first intermediates with hydrogen peroxide to form second intermediates each having the structure of Formula (Vb): 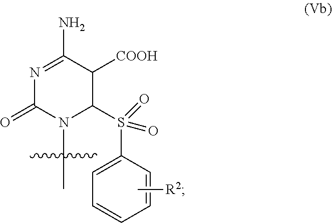
reacting the second intermediates with 1,8-diazabicyclo[5.4.0]undec-7-ene (DBU) to convert the second intermediates to uracil moieties to form a modified nucleic acid sequence; and
amplifying the modified nucleic acid sequence.

22. The method of claim 20 or 21, further comprising:
sequencing the amplified modified nucleic acid sequence; and
determining the sites of converted uracil moieties by comparing the modified nucleic acid sequence to a reference nucleic acid sequence.

23. A method of identifying cytosine methylation of a nucleic acid sequence in a nucleic acid sample, comprising:
contacting the nucleic acid sample with a TET enzyme to convert methylated cytosines and hydroxymethylated cytosines in the nucleic acid sequence to carboxylated cytosines;
reacting carboxylated cytosines in the TET treated nucleic acid sample with an unsaturated reagent in a cycloaddition reaction to convert carboxylated cytosines to first intermediates each having the structure of Formula (VI): 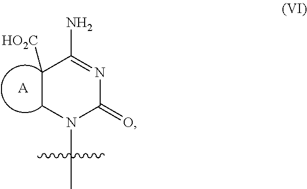
wherein ring A is an optionally substituted 4, 5 or 6 membered carbocyclyl or heterocyclyl ring;
converting the first intermediates to bicyclic thymine moieties each having a structure of Formula (VII): 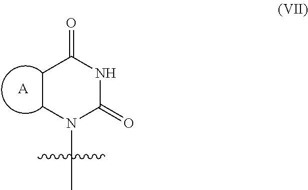
to form a modified nucleic acid sequence; and
amplifying the modified nucleic acid sequence.

24. A method of identifying methylated cytosines of a nucleic acid sequence in a nucleic acid sample, comprising:
contacting the nucleic acid sample with β-glucosyltransferase (β-GT) to selectively glucosylating hydroxymethyl cytosines of the nucleic acid sequence;
contacting the β-GT treated nucleic acid sample with a TET enzyme to convert methylated cytosines in the nucleic acid sequence to carboxylated cytosines;
reacting carboxylated cytosines in the TET treated nucleic acid sample with an unsaturated reagent in a cycloaddition reaction to convert carboxylated cytosines to first intermediates each having the structure of Formula (VI): 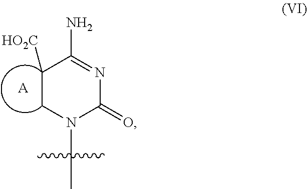
wherein ring A is an optionally substituted 4, 5 or 6 membered carbocyclyl or heterocyclyl ring;
converting the first intermediates to bicyclic thymine moieties each having a structure of Formula (VII): 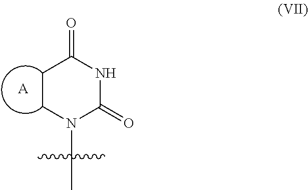
to form a modified nucleic acid sequence; and
amplifying the modified nucleic acid sequence.

25. The method of claim 23 or 24, wherein the unsaturated reagent is a 1,4-diene and the bicyclic thymine moiety having a structure of Formula (VIIa): 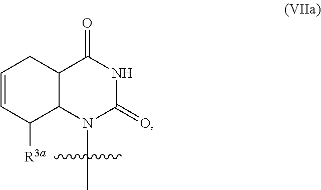
wherein R^{3a} is C₁-C₆ alkyl group optionally substituted with one or more hydrophilic moieties.

26. The method of claim 23 or 24, wherein the unsaturated reagent is an azide and the bicyclic thymine moiety having a structure of Formula (VIIb): 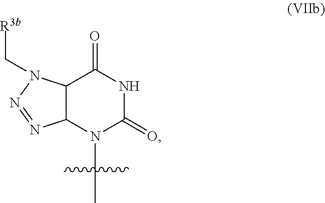
wherein R^{3b} is C₁-C₆ alkyl group optionally substituted with one or more hydrophilic moieties.

27. The method of any one of claims 23 to 26, further comprising:
sequencing the amplified modified nucleic acid sequence; and
determining the sites of bicyclic thymine moieties by comparing the modified nucleic acid sequence to a reference nucleic acid sequence.

28. The method of any one of claims 1 to 27, wherein the nucleic acid sample is a genomic DNA sample.
